(19) United States
(12) Patent Application Publication
(10) Pub. No.: US 2006/0188934 A1
(43) Pub. Date: Aug. 24, 2006

(54) System and method for implementing a high-sensitivity sensor with improved stability

(76) Inventors: Ying-Lan Chang, Cupertino, CA (US); Michael R. T. Tan, Menlo Park, CA (US)

Correspondence Address: AGILENT TECHNOLOGIES, INC.;INTELLECTUAL PROPERTY ADMINISTRATION, LEGAL DEPT., M/S DU404, P.O. BOX 7599, LOVELAND, CO, 80537-0599, US

(21) Appl. No.: 11/062,707

(22) Filed: Feb. 22, 2005

(51) Int. Cl. G01N 33/53; C12M 1/34

(52) U.S. Cl. 435/7.1; 435/287.2; 977/701

(57) ABSTRACT

A high-sensitivity sensor with improved stability includes nanostructure-based sensors that are arranged such that a first nanostructure-based sensor (“shielded sensor”) is shielded from potential exposure to an environmental factor of interest and a second nanostructure-based sensor (“exposed sensor”) is allowed potential exposure to an environmental factor of interest. Further, all of the nanostructure-based sensors are arranged to allow common exposure to environmental factors not of interest. Thus, relative changes in properties, such as electrical resistance, of the shielded nanostructure-based sensor versus changes in properties of the exposed nanostructure-based sensor are used for detecting an environmental factor of interest.

### BACKGROUND OF THE INVENTION

Semiconductor nanowires and nanotubes represent an important class of nanostructured materials with the potential to impact applications from nanoscale electronics to biotechnology. Nanostructures, such as nanowires and nanotubes, can be used as highly sensitive sensors. For example, the significant conductance change of single-walled carbon nanotubes in response to the physisorption of ammonia and nitrogen dioxide demonstrates their ability to act as extremely sensitive gas-phase chemosensors, see Qi, P. et al., Nano Lett. 3, 347 (2003). Such sensitivity has been demonstrated to be transferable to the aqueous phase for small biomolecule and protein detection in physiological solutions. That is, binding of proteins to the surface of carbon nanotube devices, or to a suitable binding receptor immobilized on the devices, results in a conductance change as well, see Robert J. Chen et al., JACS 126(5), 1563-1568 (2004). The possibility of using silicon (Si) nanowires for probing small molecule-protein interactions has also been recently demonstrated, see J. Hahm, C. M. Lieber, Nano Lett. 4(1), 51-54 (2004), and F. Patolsky et al., PNAS, Vol. 101, No. 39, 14017-10422 (2004).

Nanowires and nanotubes have been used in forming field effect transistors (FETs). One approach for the fabrication of nanowires and nanotubes into FETs is to deposit nanowires/nanotubes on thermal SiO₂, followed by metal contact formation. The FETs can then be used as sensors, such as gas-phase chemosensors. Due to their small size (e.g., nanowires typically have a diameter of approximately 30 nanometers or less), the nanostructures, such as nanowires and nanotubes, are highly sensitive to changes in the environment to which they are exposed. Thus, for example, nanostructures may be able to detect the presence of very few (e.g., even a single) molecules that are of interest. For instance, the electrical properties, such as the electrical resistance, of the nanowires/nanotubes forming a FET change as a molecule binds with them, and such a change in the resistance across the FET may enable detection of the presence of such molecules. As mentioned above, the nanowires/nanotubes may have their surfaces coated with a receptor for a particular molecule that is of interest, which enables detection of such particular molecule. Use of receptors for functionalizing the nanostructures enables selectivity as to the particular molecule to bind to the nanostructure.

However, the high sensitivity of nanostructures, as well as the nanostructure/metal interface in FETs, to environmental factors (e.g., temperature, etc.) can affect the stability of the devices in which they are implemented, thereby effecting the response to conditions that are of interest. For instance, as mentioned above, nanostructures may be used in forming FETs, which may act as chemosensors for detecting the presence of a particular molecule by detecting a change in electrical resistance in the nanostructure, which is presumed to indicate binding of the particular molecule that is of interest to the nanostructure. However, because the nanostructure is highly sensitive to other environmental factors that are not of interest, such as changes in temperature, such other environmental factors that are not of interest may cause a change in the resistance of the nanostructure, thus resulting in a false-positive indication by the chemosensor. That is, an environmental factor not of interest, such as temperature, may cause a nanostructure's electrical properties to change in a manner that may be mistaken for sensing of the environmental factor of interest, such as presence of a particular molecule. Thus, the high sensitivity of nanostructures that renders such nanostructures attractive for many sensing applications also renders the nanostructures unstable as many different environmental factors can affect the properties, such as the electrical properties of the nanostructures that are being used for sensing something that is of interest.

### BRIEF SUMMARY OF THE INVENTION

In view of the above, a desire exists for a system and method that enable high-sensitivity sensors. As described above, nanostructure-based sensors provide high sensitivity. However, due to their high sensitivity, such nanostructure-based sensors have traditionally been unstable. That is, nanostructure-based sensors are sensitive to environmental factors that are not of interest in addition to those environmental factors that are of interest. This sometimes results in false-positive signals or other errors in the output of the sensors. Thus, a further desire exists for a system and method that enable high-sensitivity sensors that are stable. That is, it is desirable to provide nanostructure-based sensors that have high sensitivity for detecting an environmental factor of interest, while being insensitive to environmental factors that are not of interest.

Novel systems and methods are provided herein for implementing a high-sensitivity sensor with improved stability. According to various embodiments provided herein, nanostructure-based sensors are arranged such that at least one of the nanostructure-based sensors (“shielded sensors”) is shielded from potential exposure to an environmental factor of interest, and at least one of the nanostructure-based sensors (“exposed sensors”) is arranged to allow potential exposure to an environmental factor of interest. Further, all of the nanostructure-based sensors are arranged to allow common exposure to environmental factors that are not of interest. Thus, relative changes in properties of the shielded sensor(s) versus changes in properties of the exposed sensor(s) can be used for detecting an environmental factor of interest. That is, because all of the nanostructure-based sensors are exposed to environmental factors that are not of interest, those factors will cause uniform changes in the monitored property(ies), such as resistance, of nanostructures of both the shielded and the exposed sensors. Whereas, because only the exposed sensors can potentially encounter the environmental factor of interest, exposure to such environmental factor of interest will cause a change in the monitored property(ies) of the nanostructures of the exposed sensors without causing a uniform change in the nanostructures of the monitored property(ies) of the shielded sensors. Thus, such a change in the monitored property(ies) of the nanostructures of the exposed sensors without a uniform change in the monitored property(ies) of the nanostructures of the shielded sensors provides an accurate indication that the environmental factor of interest has been detected by the sensors. Additionally, this sensor embodiment is very stable. That is, changes in monitored property(ies) of the nanostructures due to environmental factors that are not of interest are uniformly encountered by the nanostructures of both the exposed and the shielded sensors, and such a uniform change indicates that the changes are not due to detection of the environmental factor of interest, thus minimizing or alleviating false-positives and other errors in the output signals. In this regard, certain embodiments effectively balance or calibrate the shielded and exposed sensors across changes in environmental factors that are not of interest.

According to one embodiment, nanostructure-based sensors are arranged to form a bridge, such as a Wheatstone bridge. At least one sensor on one side of the bridge is an exposed sensor and at least one sensor on the opposite side of the bridge is a shielded sensor. The flow of current across the bridge can be monitored to detect the exposed sensor encountering the environmental factor of interest. For example, the resistance of the sensors on each side of the bridge may be initially balanced such that no current flows across the bridge. The resistance (of the nanostructures of each sensor) may change uniformly responsive to common exposure to environmental factors that are not of interest, thus maintaining no current flow across the bridge. That is, the opposing sides of the bridge remain in balance across changes in environmental factors that are not of interest. However, exposure to an environmental factor of interest results in a change in resistance in the nanostructure of the exposed sensor on one side of the bridge without a uniform change in the nanostructure of the shielded sensor(s) on the opposite side of the bridge, and thus current flows across the bridge. That is, the bridge becomes imbalanced upon exposure to the environmental factor of interest. Accordingly, such flow of current across the bridge can be detected and used as an indication that the environmental factor of interest has been detected.

### BRIEF DESCRIPTION OF THE DRAWINGS

FIG. 1 shows an exemplary sensing system according to one embodiment of the present invention;

FIG. 2A shows an exemplary implementation of a nanostructure-based sensor that may be utilized in accordance with embodiments of the present invention;

FIG. 2B shows an example of a change in resistance of the nanostructure of FIG. 2A resulting from binding of a molecule with such nanostructure;

FIG. 3 shows an exemplary nanostructure-based sensor configuration in which the surface of the nanostructure is functionalized for selectively binding to particular molecules that are of interest;

FIGS. 4A-4F show a first exemplary fabrication technique that may be utilized for forming a nanostructure-based sensor;

FIGS. 5A-5B show another exemplary fabrication process for forming a nanostructure-based sensor;

FIG. 6 shows an exemplary flow diagram for forming a high-sensitivity sensing system according to one embodiment of the present invention; and

FIG. 7 shows an operational flow diagram of a high-sensitivity sensing system according to one embodiment of the present invention.

### DETAILED DESCRIPTION OF THE INVENTION

Systems and methods are described herein for implementing a high-sensitivity sensing system with improved stability. According to various embodiments provided herein, nanostructure-based sensors are utilized for forming the high-sensitivity sensing system. In certain implementations, each nanostructure-based sensor contains one or more nanowires (or nanotubes). While nanowires and nanotubes are used in describing the exemplary embodiments herein, other nanostructures (particularly those having high aspect ratios), such as nanofibers, nanoribbons, nanothreads, nanorods, nanobelts, nanosheets, and nanorings, as examples, may be used in forming sensors. These and other nanostructures that may be used in sensors are known in the art, and future-developed nanostructures may likewise be used. Thus, as used herein, “nanostructure” broadly encompasses any of the above-mentioned and future-developed structures having at least one dimension that is of a nanoscale-size. As described above, such utilization of nanostructures enables highly sensitive sensors.

The nanostructure-based sensors are arranged such that at least one of the nanostructure-based sensors (“shielded sensor”) is shielded from potential exposure to an environmental factor of interest, and at least one of the nanostructure-based sensors (“exposed sensor”) is arranged to allow potential exposure to an environmental factor of interest. For instance, in one embodiment the shielded sensor may be covered with a covering layer, such as a Si or insulator layer, while the exposed sensor is left uncovered. Further, all the nanostructure-based sensors are arranged to allow common exposure to environmental factors that are not of interest. For instance, in one embodiment both the shielded sensor and the exposed sensor are exposed to certain environmental factors not of interest.

For example, suppose that a certain type of molecule (e.g., a toxic molecule) is an environmental factor of interest. In one embodiment, the shielded sensor is covered with a covering layer such that it cannot encounter any such molecule that may be present in the environment, while the exposed sensor is left uncovered for potential exposure to a molecule that may be present in the environment. Both the shielded and exposed sensors are exposed to temperature conditions and humidity conditions, as examples, of the environment. Thus, because both shielded and exposed sensors are exposed to these environmental factors not of interest, any change in the sensors' properties (e.g., electrical properties) resulting from such environmental factors not of interest will be uniformly experienced by both the shielded and exposed sensors. However, upon encountering the environmental factor of interest (the toxic molecule in this example), the exposed sensor experiences a change in its properties (e.g., electrical properties) without a uniform change being experienced by the shielded sensor.

Accordingly, relative changes in properties, such as electrical resistance, of the shielded sensor versus changes in properties of the exposed sensor can be used for detecting an environmental factor of interest. A change in the monitored property(ies) of the exposed sensor without a uniform change in the monitored property(ies) of the shielded sensor provides an accurate indication that the environmental factor of interest has been detected by the sensing system. Additionally, this embodiment of a sensing system is very stable. That is, changes in monitored property(ies) of the nanostructure-based sensors due to environmental factors not of interest occur uniformly in both the exposed sensor and the shielded sensor, and such a uniform change indicates that the changes are not due to detection of the environmental factor of interest, thus minimizing or alleviating false-positives and other errors in the output signals.

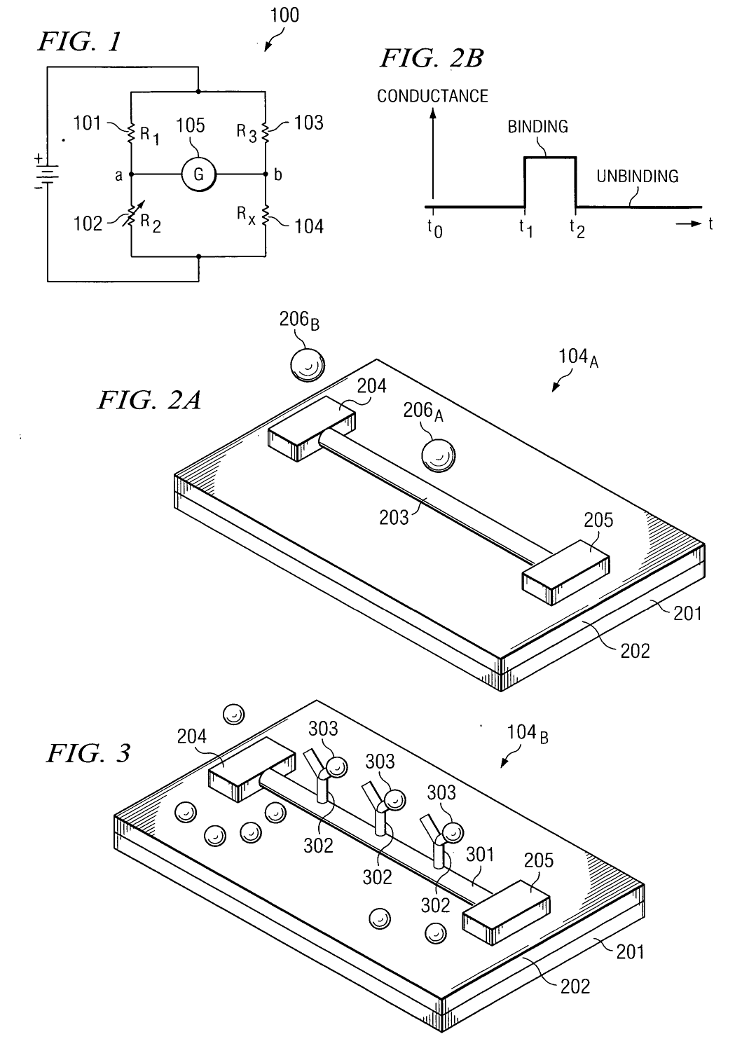

Turning to FIG. 1, an exemplary sensing system 100 according to one embodiment of the present invention is shown. This exemplary sensing system 100 includes nanostructure-based sensors 101, 102, 103, and 104. That is, each of sensors 101-104 includes a nanostructure for sensing. In certain implementations, each sensor 101-104 may contain one or more nanostructures. In the example of FIG. 1, sensors 101-104 are each resistance-based sensors having resistances R₁, R₂, R₃, and Rₓ, respectively.

In exemplary sensing system 100, sensors 101-103 are shielded from an environmental factor of interest for sensing, while sensor 104 is exposed such that it is capable of encountering the environmental factor of interest. For example, sensors 101-103 may be covered with a covering layer, or otherwise shielded, such that they cannot encounter an environmental factor of interest (e.g., a given molecule of interest), while sensor 104 may be left uncovered. Thus, in this example, sensors 101-103 are shielded sensors and sensor 104 is an exposed sensor. Sensors 101-104 are all similarly exposed to environmental factors that are not of interest for sensing. Any suitable technique now known or later developed for shielding sensors 101-103 from the environmental factor of interest, while leaving sensors 101-104 all similarly exposed to environmental factors that are not of interest for sensing may be employed in embodiments of the present invention. In certain embodiments, the shielded sensors 101-103 may be implemented such that they are not receptive to an environmental factor of interest, while the exposed sensor 104 is implemented to be receptive to such an environmental factor of interest. For example, the nanostructure of the exposed sensor 104 may be coated with a receptor so as to adapt the nanostructure to be receptive to an environmental factor of interest (e.g., a given molecule of interest), whereas the shielded sensors 101-103 are not so adapted to be receptive to the environmental factor of interest and are thus effectively shielded from receiving such environmental factor of interest but all of such sensors are exposed to environmental factors not of interest. Accordingly, in certain embodiments a physical shield (e.g., cover) is used to shield the shielded sensors, and in other embodiments, instead of being physically shielded, the shielded sensors may not be adapted to be responsive to an environmental factor of interest (e.g., not coated for receiving a molecule of interest) while the exposed sensor is so adapted. Thus, except where specified otherwise herein, the term “shield” is intended to encompass any of these techniques of shielding a sensor.

Suppose, for example, that it is desired to sense the presence of a particular molecule of interest within an environment in which the sensing system 100 of FIG. 1 is placed. In this case, the nanostructure of sensor 104 may be coated with a receptor for the molecule of interest, such as described further below in the example of FIG. 3. The nanostructure of sensor 104 is exposed to the environment in a manner such that it can encounter the molecules of interest that may be present in the environment, while the nanostructures of sensors 101-103 are shielded from potential exposure to the molecules of interest that may be present in the environment. However, the nanostructures of sensors 101-104 are commonly exposed to other environmental factors that are not of interest, such as temperature of the environment.

This exemplary sensing system 100 allows one to monitor very small changes in resistance Rₓ resulting from changes in the surface of the nanostructure of exposed sensor 104. The resistances R₁, R₂, and R₃ of the shielded sensors 101-103 are used to “balance” other resistance changes due to environmental factors that are not of interest, such as temperature in the above example. In this example, sensors 101-104 are electrically connected to form a bridge of the type commonly referred to as a Wheatstone bridge. That is, sensors 101 and 102 form a first voltage divider and sensors 103 and 104 form a second voltage divider. The current flow between points a and b in sensing system 100 can be monitored with a current sensor, such as a galvanometer 105. If the two voltage dividers have exactly the same ratio (R₁/R₂=R₃/Rₓ), then the circuit is said to be balanced and no current flows in either direction through galvanometer 105. If the resistance of one of the sensors 101-104 changes, even by a small amount, relative to the resistance of the other sensors, the circuit will become unbalanced and current will flow through galvanometer 105. Thus, galvanometer 105 provides a very sensitive indication of the balance condition.

It should be recognized that because the sensors 101-104 are all similarly exposed to environmental factors that are not of interest for sensing, such as temperature in the above example, changes in the environmental factors that are not of interest will be encountered by all of the sensors 101-104, and thus like conditions will be experienced by each of the sensors and the circuit will remain balanced. However, because sensors 101-103 are shielded from an environmental factor of interest for sensing, such as the molecule of interest in the above example, while sensor 104 is exposed to such environmental factor of interest for sensing, sensor 104 can encounter the environmental factor of interest without sensors 101-103 encountering such environmental factor of interest. Thus, a change in sensor 104's electrical properties, such as resistance value Rₓ of sensor 104, occurs upon exposure to the environmental factor of interest without a uniform change in the electrical properties of sensors 101-103. This will result in an imbalance in the circuit. Thus, the sensing system 100 provides a high degree of assurance that an imbalance of the circuit is a result of detection of the environmental factor of interest, rather than being due to some environmental factor not of interest. Accordingly, the exemplary sensing system 100 provides high sensitivity and has improved stability over prior high-sensitivity sensing systems.

In practical application of one embodiment, the values of the resistances R₁ and R₃ of sensors 101 and 103 are precisely known, but do not have to be identical. The resistance R₂ of sensor 102 can initially be calibrated variable resistance, and the value of the variable resistance may be read from a scale, for example. The resistance of a nanostructure can be adjusted by applying a voltage to such nanostructure. The resistance R₂ of sensor 102 is initially adjusted until galvanometer 105 reads zero current. At this point,

$${R}_{x} = \frac{{R}_{2} \times {R}_{3}}{{R}_{1}} .$$

The resistance Rₓ of sensor 104 can then be determined and compared before and after exposure to the environment of interest. In this exemplary embodiment, the bridge is initially balanced such that no current is flowing across it so that a change occurring only in the resistance Rₓ is easily detected because it causes current to flow across the bridge (i.e., the bridge becomes unbalanced). So, the value of resistance Rₓ of sensor 104 when not encountering the environmental factor of interest, such as the particular molecule in the above example, can initially be determined. Thus, the circuit can initially be calibrated such that R₁/R₂=R₃/Rₓ, and then the circuit can be used for sensing the environmental factor of interest. The resistance of a nanostructure-based sensor can be adjusted in this calibration by applying a bias to the sensor. As described above, an environmental factor not of interest will have a like effect on the electrical properties of all of the sensors 101-104 as all of the sensors 101-104 are exposed to such environmental factor not of interest. Thus, the sensing system 100 will remain substantially in balance when encountering changes in the environmental factor not of interest. However, sensor 104 is capable of encountering an environmental factor of interest, while sensors 101-103 are shielded from exposure to such environmental factor of interest. Accordingly, upon the sensing system 100 encountering the environmental factor of interest, the electrical properties of sensor 104 will change relative to those of sensors 101-103, thus causing an imbalance in the sensing system. Because the sensing system 100 remains substantially in balance when encountering changes in the environmental factor not of interest, an imbalance in the sensing system is a very good indicator of detection by sensor 104 of the environmental factor of interest.

Instead of trying to adjust resistance R₂ of sensor 102 to balance the sensing system 100, the galvanometer 105 can, in certain embodiments, be replaced by a circuit that can be used to record the imbalance in sensing system 100 as sensor 104 is exposed to an environment. The circuit can also be designed in a way that will automatically “re-zero” at the starting point. In this sense, the “starting point” refers to the beginning of the measurement of interest. That is, starting point refers to a time before the environmental factor of interest (e.g., a biomolecule of interest) is introduced. The circuit may have a non-zero reading to start with due to variations among sensors, as discussed further herein. One can record the initial value, and compare it with the final value. Or, one can design a circuit to “reset” the sensing system so that the initial value is zero, and then display the difference after sensor 104 is exposed to the environmental factor of interest. Resetting the sensing system simply means that the reading will be set to zero, no matter what the current value is.

The exemplary sensing system 100 of FIG. 1 has several advantages over prior sensing systems incorporating nanostructure-based sensors for sensing. First, sensors 101-104 are made with similar structures and material. Thus, they have similar characteristics. As a result, when connected as shown in FIG. 1, their response to an environmental factor not of interest, such as temperature in the above example, cancels out. Also, in this case, a voltage difference (not an absolute voltage value) is measured. Further, the fact that resistance R₁ of sensor 101 and resistance R₃ of sensor 103 do not need to be identical allows some degree of differences in the nanostructures implemented in sensors 101 and 103. For example, as-grown nanotubes can be a mixture of semiconducting/metallic tubes, which may result in differences in resistance in the nanotubes implemented for different sensors. As another example, differences in the diameters of the nanotubes and nanowires can affect their device characteristics. This can apply to all the sensors 101-104. Basically, any differences among the intrinsic characteristics of nanotubes or nanowires can be canceled out by setting the initial value of resistance R₂ of sensor 102. The initial value of resistance R₂ can be set by applying a bias to the nanostructure of sensor 102. In certain implementations, the monitored output of sensing system 100 is the voltage difference across the bridge. Such voltage differential can be amplified by another circuit (not shown in FIG. 1) to further enhance the sensitivity, if so desired. In certain embodiments, the voltage differential may be measured to determine how much, if any, such voltage differential has changed, and if the amount of change in the voltage differential is more than a threshold, it indicates the presence of the environmental factor of interest.

While an exemplary sensing system 100 is shown in FIG. 1, embodiments of the present invention are not limited to this specific configuration. For instance, while four sensors 101-104 are shown in FIG. 1, other sensing systems may include a different number of sensors (e.g., may include more or fewer than four), with at least one sensor arranged to be exposed to an environmental factor of interest and at least one sensor shielded from exposure to such environmental factor of interest. As another example, sensing system 100 of FIG. 1 may be modified to omit sensors 102 and 103, thus leaving shielded sensor 101 and exposed sensor 104. In this case, resistance R₁ of shielded sensor 101 may be used as a reference to determine whether resistance Rₓ of exposed sensor 104 changes uniformly with resistance R₁ of sensor 101. Thus, the difference of Rₓ−R₁ may be recorded over time and used for indicating when an environmental factor of interest has been detected. For instance, if the difference of Rₓ−R₁ remains constant, the environmental factor of interest is not detected. However, if the difference of Rₓ−R₁ changes, this indicates that something has changed the resistance Rₓ of exposed sensor 104 without similarly changing the resistance R₁ of shielded sensor 101, thus indicating detection of the environmental factor of interest.

FIG. 2A shows an exemplary implementation of a nanostructure-based sensor that may be utilized in accordance with embodiments of the present invention. The exemplary sensor shown in FIG. 2A is labeled 104_{A}, as it may be implemented for sensor 104 of the exemplary embodiment of FIG. 1. In such an implementation, sensors 101-103 of FIG. 1 would be similar nanostructure-based sensors. Of course, the exemplary embodiment of FIG. 1 is not limited to use of the exemplary nanostructure-based sensor 104_{A} of FIG. 2A, but may additionally or alternatively include other nanostructure-based sensors that are now known or later developed. Sensor 104_{A} is one type of nanostructure-based sensor that is known in the art. Such sensor 104_{A} is effectively a field-effect transistor (FET) 104_{A} formed with a nanowire that couples a source and a drain. Sensor 104_{A} includes backgate 201; oxide layer 202; nanowire 203, which is shown in this example as a Si nanowire; source 204; and drain 205. As described further below, fabrication techniques are known for forming this, as well as other implementations, of nanostructure-based FETs.

As further illustrated in FIG. 2A, the FET 104_{A} may be used as a sensor, such as a chemosensor. For instance, as molecules 206A and/or 206B bind with the surface of nanowire 203, the electrical properties, such as the resistance, of such nanowire 203 change. This change in the electrical properties of nanowire 203 can be measured by, for example, measuring the resistance change. The waveform in FIG. 2B illustrates an example in which the resistance of nanowire 203 of FIG. 2A is shown over a period of time. Initially, at time t₀, the resistance of nanowire 203 is at a first level. Such resistance level remains steady until time t₁, at which binding of a molecule 206A and/or 206B with the surface of nanowire 203 occurs. As the waveform of FIG. 2B illustrates, such binding of the molecule with the surface of nanowire 203 changes the resistance of nanowire 203. Detecting such change in the resistance of nanowire 203 may be used for sensing the presence of molecules 206A and 206B. At time t₂, the molecule unbinds from the surface of nanowire 203, and thus the resistance of nanowire 203 returns to its initial level.

In view of the above, in certain embodiments, the nanostructure-based sensors may each be implemented as a nanostructure-based FET having a molecular gate. Instead of applying bias on the gate electrode, in this case, the surface charge can be introduced as a result of molecular attachment, for example. For instance, the charge introduced as a result of molecular attachment (i.e., attachment of a molecule of interest) causes a change of channel width, and therefore a change in the conductance of the channel.

Of course, while the resistance is shown in this example as steady except when binding occurs, various other environmental factors may affect the resistance of nanowire 203. For instance, changes in temperature, moisture, humidity, and/or presence of gases that are not of interest in the environment will cause a change in the resistance of nanowire 203. The simplified example of FIG. 2B assumes that all environmental factors remain constant, except for the binding of molecule 206A/206B with nanowire 203. For many applications, it is desirable to utilize a high-sensitivity sensor in environments in which various factors that are not of interest may change, e.g., in an “uncontrolled environment”. As one example, an individual or robot may be equipped with a high-sensitivity sensor for detecting the presence of certain molecules of interest, e.g., toxic molecules, as the individual/robot moves about an open and/or relatively uncontrolled environment. For example, the robot may move about a city, a given building, etc, in which various environmental factors that are not of interest, such as temperature in this example, may change. It is thus desirable that the high-sensitivity sensor be implemented such that it remains highly sensitive to an environmental factor of interest, e.g., toxic molecules, but remain stable with respect to changes in an environmental factor not of interest.

As described with reference to the exemplary sensing system 100 of FIG. 1, embodiments of the present invention enable high-sensitivity sensors to be employed in a manner that cancels out changes in the sensors resulting from environmental factors not of interest, thus allowing a stable, highly-sensitive sensor that accurately detects the environmental factors of interest while reducing/eliminating false positive detections. More particularly, embodiments provided herein include nanostructure-based sensors (such as sensors 101-104 of FIG. 1), wherein at least one of the sensors (e.g., sensor 104 of FIG. 1) is exposed to the environment to enable it to encounter an environmental factor of interest, for instance, toxic molecules in the above example, while at least one other of the sensors, e.g., sensors 101-103 of FIG. 1, is shielded from encountering such environmental factor of interest. For instance, a SiN or oxide layer, or even a polymer-based passivation layer, as examples, may be deposited over sensors 101-103 of FIG. 1 to shield them from encountering molecules 206A and 206B. On the other hand, sensor 104 remains uncovered so that it is exposed to any such molecules 206A and 206B that may be present in the environment in which the sensor is placed. All of the sensors are commonly exposed to various environmental factors not of interest, for instance, temperature in the above example. As such, the electrical properties of the sensors can be compared to determine whether they are changing together, thus indicating exposure to an environmental factor not of interest, or if the exposed sensor is changing without a like change in the shielded sensor(s), thus indicating exposure to the environmental factor of interest.

In accordance with various embodiments described herein, properties, e.g., electrical properties, of both shielded and exposed sensors will change “uniformly” when exposed to an environmental factor not of interest. In this case, changing “uniformly” does not mean that the properties of the nanostructures of each sensor will necessarily change identically, but they will change similarly and in the same sense (e.g., a 5% increase in resistance, a 2% decrease in resistance, etc.). For instance, the sensors may be typically operated in ranges in which they will have a linear response. As an example, in certain implementations a nanowire implemented in a first sensor may be slightly longer than a nanowire implemented in a second sensor. Thus, the changes in the properties of the two nanowires when they both encounter a given environmental factor, e.g., a change in temperature, may differ. As another example, the diameters and/or doping levels of the nanowires in the first and second sensors may differ. Thus, the changes in the properties of the nanowires when they both encounter a given environmental factor may differ. However, while the changes in the properties of the nanowires may differ, the properties of both nanowires will change uniformly in the sense that the property, e.g., electrical resistance, will either increase or decrease in response to a commonly encountered environmental factor. Additionally, the changes in the properties of the two nanowires resulting from the commonly encountered environmental factor will be proportional.

On the other hand, the “exposed” sensor detecting an environmental factor of interest will result in the properties, e.g., electrical properties of the nanostructure of such exposed sensor, changing non-uniformly relative to those of the nanostructure of the shielded sensor. Thus, if the properties of the nanostructure of the exposed sensor change non-uniformly relative to the properties of the nanostructure of the shielded sensor, such a non-uniform change indicates that the exposed nanostructure has encountered an environmental factor not encountered by the shielded nanostructure, which as described above is a good indication that the environmental factor of interest has been detected.

FIG. 3 shows an exemplary nanostructure-based sensor configuration in which the surface of the nanostructure is functionalized for selectively binding to particular molecules of interest. In this sense, “functionalized” refers to treating the nanostructure's surface with specific functional group so that its surface can only react to the specific molecules of interest. In this sense, the surface reacts by formation of a bond and charge transfer with the specific molecules of interest. The charge transfer modulates the channel width, and therefore the conductance. The exemplary sensor shown in FIG. 3 is labeled 104_{B}, as it may be implemented for sensor 104 of the exemplary embodiment of FIG. 1. In such an implementation, sensors 101-103 of FIG. 1 would be similar nanostructure-based sensors (which may or may not have their surfaces functionalized in a like manner), but are shielded such that they do not react to the specific molecules of interest. The exemplary sensor 104_{B} includes a FET 104_{B} as described in the example of FIG. 2A. That is, the FET 104_{B} includes backgate 201, oxide layer 202, source 204, and drain 205. The exemplary FET of FIG. 3 includes a nanowire 301 that couples source 204 with drain 205, wherein nanowire 301 has its surface functionalized with antibodies 302. In this manner, nanowire 301 is functionalized for selectively binding with antigens 303. That is, nanowire 301 is functionalized to encourage binding with certain molecules (antigens 303), while discouraging binding with other molecules. Various techniques are known for coating the surface of a nanostructure (e.g., nanowire) for encouraging binding of selected molecules are known, and any such technique now known or later developed for functionalizing the surface of a nanostructure for binding with any molecule that may be of interest for detection may be utilized.

It should be recognized that binding antibodies 302 with the surface of nanowire 301 will likely change the electrical properties (e.g., resistance) of the nanowire. However, when antigens 303 bind with antibodies 302, the electrical properties (e.g., resistance) of the nanowire will further change. Thus, in this exemplary embodiment, the electrical properties of nanowire 301 having antibodies 302 bound thereto are calibrated with the electrical properties of other, shielded nanostructure-based sensors. For instance, if sensor 104_{B} of FIG. 3 is implemented in place of sensor 104 of FIG. 1, sensors 101-103 may be calibrated such that R₁/R₂=R₃/Rₓ after antibodies 302 are bound to the surface of nanowire 301. That is, the bridge is balanced to account for the electrical properties of nanowire 301 having antibodies 302 bound thereto. Thus, upon antigens 303 binding with antibodies 302, the further change in the electrical properties of nanowire 301 results in an imbalance in the circuit (as the nanostructures of sensors 101-103 are shielded from binding with antigens 303), thus enabling detection of such antigens 303.

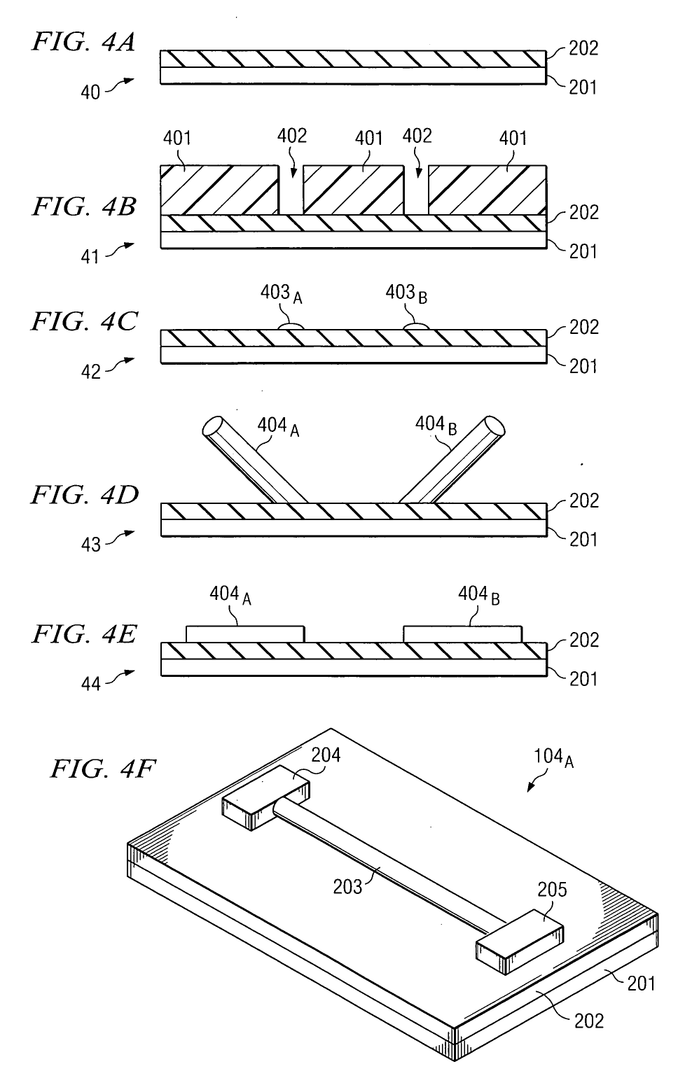

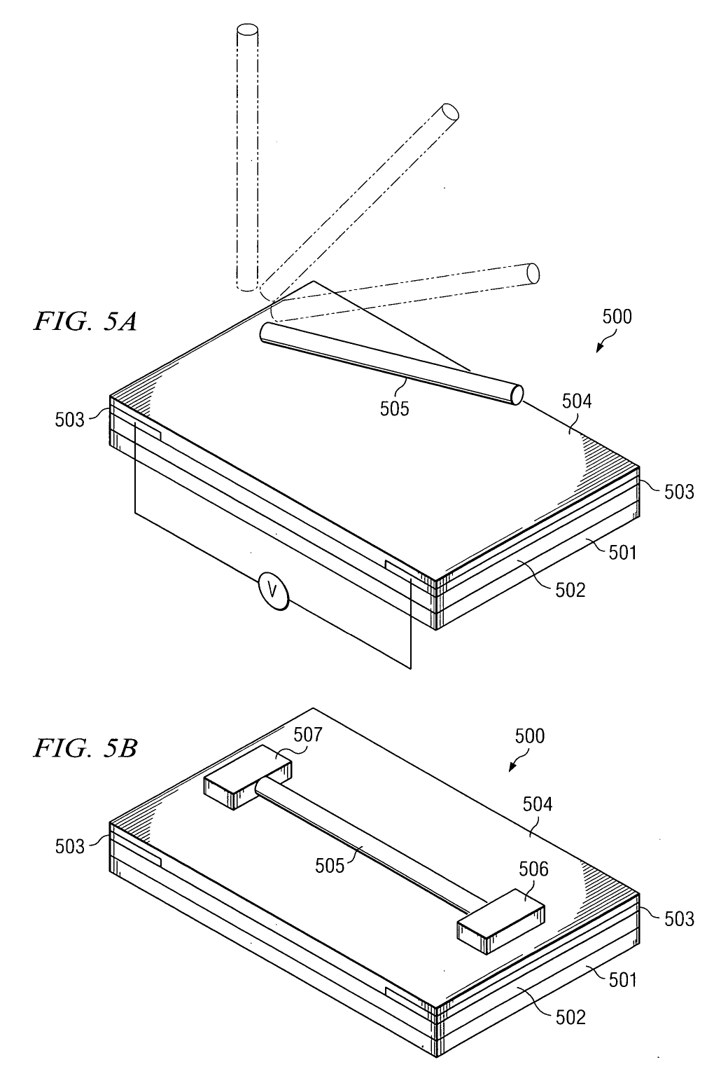

Any fabrication technique now known or later developed for fabricating nanostructure-based sensors, such as those of FIGS. 2 and 3, arranged in a sensing system such as that of FIG. 1 may be employed. Exemplary fabrication techniques that may be utilized are described further below in connection with FIG. 4-6. However, embodiments of the present invention are not intended to be limited to circuits fabricated utilizing any particular fabrication technique, but instead the exemplary fabrication techniques are provided herein merely for illustrative purposes and to make evident that the embodiments described herein can be fabricated and are thus enabled by this disclosure.

As described above, one type of nanostructure-based sensor is a FET in which a nanotube, e.g., a semiconducting carbon nanotube, or nanowire is used as the channel. The source and drain are typically metal layers connected to the nanotube/nanowire. The nanotubes/nanowires are typically added into the device by either direct growth on the substrate or by dispersion from a suspension onto the substrate.

In certain nanostructure-based FET devices, the substrate, e.g., a thermal SiO₂ layer on heavily doped silicon, on which carbon nanotubes (CNTs) were grown acts as the gate. In this so-called “back-gate” device, the silicon is the gate electrode (e.g., backgate 201 of FIGS. 2 and 3) and SiO₂ is the gate insulator (e.g., oxide layer 202 of FIGS. 2 and 3), see e.g., S. J. Tans, A. R. M. Verschueren, C. Dekker, “Room-temperature transistor based on a single carbon nanotube,” Nature, 393(7), p. 49 (1998).

Fabrication techniques are also known for making a top-gate FET that has a CNT as its channel, see e.g., S. J. Wind et al., “Vertical scaling of carbon nanotube field-effect transistors using top gate electrodes,” Appl. Phys. Lett., 80(20), p. 3817 (2002), and A. Jarvey et al., “High-k dielectrics for advanced carbon nanotube transistors and logic gates,” Nat. Mater., 1, p. 241 (2002). This top-gate FET is fabricated by deposition of the gate insulator and then deposition and patterning of the gate metal all after the CNTs are grown or dispersed on the wafer. Certain fabrication techniques are also known for making FETs with a combination of back-gate and top-gate, where the back-gate is used to increase conductance of the unmodulated tube regions between the gate and source and between gate and drains.

Below, exemplary fabrication methods that utilize nanowires in forming sensors (e.g., FETs) are further described. More particularly, exemplary direct growth methods and postgrowth assembly methods are each described.

As an example of using a direct growth method, the location of a metal catalyst for nanowire growth is defined by e-beam lithography or imprint lithography. The as-grown nanowires are typically randomly oriented. Postgrowth ion treatment is used to align the orientation of nanowires, such as that described further in U.S. Pat. No. 6,248,674 titled “METHOD OF ALIGNING NANOWIRES.” After the alignment of the nanowires, metal contact and circuit can be defined using lithography.

As an example of using a postgrowth assembly, nanostructures are grown and removed from substrates. Electrically isolated interdigitated electrodes are defined on an SiO₂/Si substrate by standard photolithography. The substrate is placed in a suspension containing nanowires and nanotubes. An alternating current (AC) voltage applied between the electrodes “attracts” the nanowires or nanotubes in the suspension. When the nanowires or nanotubes form a bridge between the electrodes, the voltage difference between the electrodes falls to zero. The alignment process is therefore self-limiting, see e.g., Smith et al., Appl. Phys. Lett, 77(9), p. 1399 (2000).

A first exemplary fabrication technique that may be utilized for forming a nanostructure-based sensor (in the example shown, a FET with a nanowire channel) is shown in FIGS. 4A-4F. In process 40 of FIG. 4A, the fabrication process begins with a degenerately-doped Si wafer with an insulator layer, thus resulting in a wafer having silicon layer 201 and insulator layer 202. A layer 401 of photo resist is deposited and E-beam lithography is utilized in process 41 of FIG. 4B to pattern the photo resist to define channels 402. In process 42 of FIG. 4C, catalyst materials 403_{A} and 403_{B} are deposited in channels 402 by e-beam evaporation a lift-off process is performed afterwards to remove layer 401. In process 43 of FIG. 4D, the nanowire growth process is performed to grow nanowires 404_{A} and 404_{B} from catalysts 403_{A} and 403_{B}, respectively. In process 44 of FIG. 4E, an alignment process, such as that described in U.S. Pat. No. 6,248,674 titled “Method of Aligning Nanowires,” is utilized to align the nanowires 404_{A} and 404_{B} as desired for a given device configuration. Finally, e-beam lithography and e-beam evaporation are utilized to deposit a metal layer and form source 204 and drain 205 from such metal layer, thereby resulting in sensor (e.g., FET) 104_{A} of FIG. 4F.

Turning to FIGS. 5A-5B, another exemplary fabrication process 500 for forming a nanostructure-based sensor (e.g., FET) is shown. In this example, the fabrication process begins, in FIG. 5A, with a degenerately-doped Si wafer 501 with insulator 502 (e.g., field oxide). Metal electrodes 503 are included which may be implemented in an interdigitated finger pattern defined by metal liftoff on a silicon dioxide (SiO₂) substrate, see “Electric-field assisted assembly and alignment of metallic nanowires” by Peter A. Smith et al., Applied Physics Letters Volume 77, Number 9, pg. 1399 (2000). The metal electrodes 503 are defined by photolithography followed by metal deposition and lift-off; The electrodes 503 are protected with a protection layer 504, such as Si₃N₄, to prevent the nanowires 505 shorting the electrodes 503 during the assembly process.

The wafer 501 having the electrodes 503 and protection layer 504 is placed in a suspension containing nanowires, and by applying alternating voltages between the electrodes 503 the nanowires, such as nanowire 505, align relative to such electrodes 503, as desired. The voltage “V” across the interdigitated finger electrodes 503 becomes 0V when the nanowire 505 is aligned across such electrodes. Once the voltage becomes 0V, no further attraction of the nanowire 505 by the interdigitated finger electrodes 503 occurs, and therefore this is a self-limiting process. The underlying electrodes 503 are simply used to define locations of nanowires. The source and drain need to be insulated from the electrodes, otherwise one would get leakage through the underlying electrodes 503.

As shown in FIG. 5B, metal contacts, such as source 507 and drain 506, are then defined in a conductive layer deposited on the protection layer 504, again using photolithography followed by metal deposition and liftoff. The source 507 and drain 506 can be aligned to the underlying electrodes 503 by designing some “alignment mark” in the mask. That is, as shown in FIG. 5A, the nanowire 505 bridges over the gap between the underlying electrodes 503. Thus, in FIG. 5B, it becomes desirable for the source 507 and drain 506 to be aligned to the underlying electrodes 503 so that the source and drain lay right on top of the nanowire 505 for forming good electrical connection to the nanowire.

This assembly technique can be used, for example, to form each of the sensors 101-104 of FIG. 1. For instance, one can define the locations of electrodes 503 based on the desired device configuration. Once the locations of the electrodes are defined, the wafer is placed in suspension containing the nanowires, and an AC voltage applied between the electrodes is used to cause nanowires to couple across the various sources/drains implemented on the wafer (as shown in FIGS. 5A-5B), thereby forming each of sensors 101-104. Assembly experiments have been conducted by dispensing a dilute suspension of nanowires or nanotubes onto samples biased with alternating electrode voltages. Alignment of nanowires has been demonstrated, suggesting that this technique may also be applied to align conductive carbon nanotubes.

Another technique for implementing an array of nanowires/nanotubes that may be employed in certain embodiments is disclosed in co-pending U.S. patent application Ser. No. 10/946,753 filed Sep. 22, 2004 and titled “SYSTEM AND METHOD FOR CONTROLLING NANOSTRUCTURE GROWTH,” the disclosure of which is hereby incorporated herein by reference. As this referenced patent application discloses, topological structures on a substrate may be utilized to influence, during growth, the arrangement of nanotubes/nanowires on such substrate. Once such nanotubes are arranged in an array, metal deposition and patterning are performed to make a source and drain to form respective FETs. Thus, FETs that each include a source, drain, and a nanostructure connected between the source and drain may be formed, and each of the FETs may be used as a sensor, where the sensors are electrically connected in a sensing system such as that of FIG. 1.

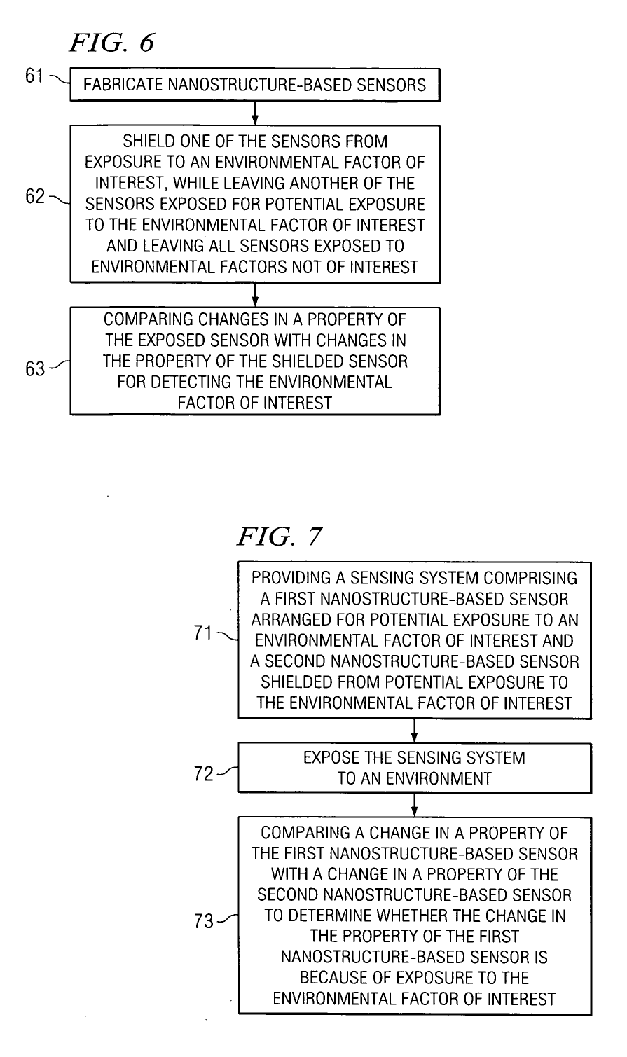

FIG. 6 shows an exemplary flow diagram for forming a high-sensitivity sensing system according to one embodiment of the present invention. In block 61, nanostructure-based sensors are provided. For example, the nanostructure-based sensors may be fabricating using, for example, any of the exemplary fabrication techniques described above. The nanostructure-based sensors include first and second nanostructure-based sensors. In block 62, one of the nanostructure-based sensors is shielded from exposure to an environmental factor of interest, while another of the nanostructure-based sensors is left exposed for potential exposure to the environmental factor of interest. Further, both of the nanostructure-based sensors are exposed to environmental factors not of interest. For instance, in the example of FIG. 2A, a sensor, such as sensors 101-103 of FIG. 1, is shielded from potential exposure to molecules 206A/206B. For example, the sensor may be shielded by covering it with, for instance, an Si or insulator layer. A sensor, such as sensor 104 of FIG. 1, is left exposed (e.g., uncovered) such that it can encounter (and nanowire 203 can bind with) any of molecules 206A/206B that may be present in the vicinity of such sensor. All of the sensors (such as sensors 101-103 of FIG. 1) are exposed to environmental factors not of interest, such as temperature in this example. Thus, environmental factors not of interest will similarly affect all the sensors, while the environmental factor of interest will affect only the sensor that is exposed to such environmental factor of interest.

In block 63, changes in a property of the exposed nanostructure-based sensor are compared with changes in the property (e.g., electrical resistance) of the shielded nanostructure-based sensor for detecting the environmental factor of interest. As described above, this comparison may be made via comparison circuitry, which may, in certain implementations, be a detector that detects flow of current, wherein the current flows when the resistance of the exposed sensor changes without the resistance of the shielded sensor similarly changing. Continuing with the above example, exposure to changes in temperature will result in a uniform change in the resistance of all the sensors, and thus because the comparison circuitry detects a uniform change among the sensors (e.g., current flow is not detected by galvanometer 105 of FIG. 1) the sensing system can determine that the change in the sensors' resistance is because of an environmental factor not of interest. In certain embodiments, rather than the comparison circuitry detecting a uniform change in properties, the uniform change in properties results in no change in the condition monitored by the comparison circuitry. For instance, in certain embodiments described above the sensors are electrically connected in a Wheatstone bridge configuration, and the current flowing across the bridge is monitored by the comparison circuitry (e.g., a galvanometer). Upon a uniform change in resistance of the sensors on both sides of the bridge, no current flow (or no change in current flow) may be detected. Thus, detection of the environmental factor of interest, such as molecules 206A/206B in this example, is not signaled by the sensor.

However, when the exposed sensor binds with molecules 206A/206B, a change in its resistance occurs without a uniform change in the resistance property of the shielded sensor occurring, and thus because the comparison circuitry detects a non-uniform change among the sensors, e.g., current flow is detected by galvanometer 105 of FIG. 1, the sensing system can determine that the change in the resistance is because of the environmental factor of interest. Thus, detection of the environmental factor of interest, such as molecules 206A/206B in this example, is signaled by the sensing system.

FIG. 7 shows an operational flow diagram of a high-sensitivity sensing system according to one embodiment of the present invention. In block 71, a sensing system is provided. The sensing system comprises a first nanostructure-based sensor arranged for potential exposure to an environmental factor of interest and a second nanostructure-based sensor shielded from potential exposure to said environmental factor of interest. In block 72, the sensing system is exposed to an environment. That is, the sensing system is exposed to an environment in which detection of an environmental factor of interest is desired. In block 73, the sensing system compares a change in a property of the first nanostructure-based sensor with a change in a property of the second nanostructure-based sensor to determine whether the change in the property of the first nanostructure-based sensor is because of exposure to the environmental factor of interest.

For instance, continuing the above example, when sensor 104_{A} of FIG. 2A is exposed to molecules 206A/206B, such molecules bind with the nanowire 203, thus changing the nanowire's resistance. The change in the property of the sensor 104_{A} is compared with an amount of change (if any) in a property of the shielded nanostructure-based sensor, such as nanostructure-based sensors 101-103 of FIG. 1. As described above, the comparison may be performed, in certain implementations, by a galvanometer that detects flow of current, wherein the current flows when the resistance of the exposed sensor changes without the resistance of the shielded sensor similarly changing. This comparison indicates whether the change in the property of the at least one exposed nanostructure-based sensor is because of exposure to the environmental factor of interest. If the resistance of the shielded sensor changes uniformly with the change in resistance of the exposed sensor, then the change in the sensors' resistance property is because of an environmental factor not of interest (such as temperature in this example). Thus, detection of the environmental factor of interest (such as molecules 206A/206B in this example) is not signaled by the sensing system. However, when the exposed sensor binds with molecules 206A/206B, a change in the exposed sensor's resistance occurs without a uniform change in the resistance of the shielded sensor occurring, and thus because the comparison detects a non-uniform change among the sensors (e.g., current flow is detected across the exemplary bridge configuration of FIG. 1), the sensing system can signal that the change in the resistance is because of the environmental factor of interest.

What is claimed is:

1. A system comprising:
a nanostructure exposed to an environment for sensing an item of interest, and a nanostructure shielded from exposure to said item of interest, wherein a monitored property of the nanostructures changes uniformly responsive to exposure to items not of interest and changes non-uniformly responsive to exposure to said item of interest.

2. The system of claim 1 wherein said nanostructures comprise one of:
nanotube, nanowire, nanofiber, nanoribbon, nanothread, nanorod, nanobelt, nanosheet, and nanoring.

3. The system of claim 1 wherein said item of interest is a molecule of interest.

4. The system of claim 3 wherein said molecule of interest is a gas molecule.

5. The system of claim 3 wherein said molecule of interest is a molecule of a liquid.

6. The system of claim 3 wherein said nanostructure exposed to said environment includes a receptor for said molecule of interest.

7. The system of claim 1 wherein said item of interest is an antigen, and wherein said nanostructure exposed to said environment includes an antibody specific to the antigen of interest.

8. The system of claim 1 wherein said items not of interest include at least one of:
temperature, moisture, humidity, and gas molecules that are not of interest.

9. The system of claim 1 wherein said nanostructures are electrically connected to form a bridge.

10. The system of claim 9 wherein said bridge comprises said nanostructure shielded from exposure to said item of interest on one side of the bridge and said nanostructure exposed to said environment on an opposite side of said bridge.

11. The system of claim 10 wherein resistances of said nanostructures on opposing sides of said bridge remain balanced except when said nanostructure exposed to said environment encounters said item of interest.

12. A system comprising:
means for sensing an item of interest, the sensing means including a first nanostructure having a property that changes responsive to sensing said item of interest; and
means for signifying whether a change in said property of the first nanostructure is because of sensing said item of interest, said signifying means including a second nanostructure shielded from exposure to said item of interest.

13. The system of claim 12 wherein the first and second nanostructures are commonly exposed to an item not of interest.

14. The system of claim 13 wherein the property of the first nanostructure and the property of the second nanostructure change uniformly responsive to common exposure to the sensed item not of interest.

15. The system of claim 12 wherein the property of the first nanostructure and the property of the second nanostructure change non-uniformly responsive to exposure of the first nanostructure to the item of interest.

16. The system of claim 15 wherein the property of the first nanostructure and the property of the second nanostructure are electrical resistance.

17. The system of claim 12 wherein the sensing means comprises a field effect transistor comprising said first nanostructure forming a channel between a source and a drain.

18. The system of claim 12 wherein the sensing means and the signifying means are connected to form a Wheatstone bridge.

19. A system comprising:
nanostructures whose electrical properties change responsive to changes in environmental factors, wherein at least one of said nanostructures is shielded from an environmental factor of interest (“shielded nanostructure”), and at least one of said nanostructures is not shielded from said environmental factor of interest (“non-shielded nanostructure”); and
said nanostructures are arranged such that exposure thereof to a common environmental factor results in similar changes in their electrical properties, and exposure of the non-shielded nano structure to said environmental factor of interest results in dissimilar a change in said electrical properties in said non-shielded nanostructure relative to the shielded nanostructure.

20. The system of claim 19 further comprising:
all of said nanostructures are exposed to environmental factors not of interest.

21. The system of claim 19 further comprising:
said nanostructures are arranged to form a bridge.

22. The system of claim 21 wherein said shielded nanostructure and said non-shielded nanostructure are on opposite sides of said bridge.

23. The system of claim 19 wherein said environmental factor of interest is one of:
a molecule of a gas of interest, a molecule of a liquid of interest, and an antigen.

24. The system of claim 19 wherein said common environmental factor to which said nanostructures are exposed includes at least one of:
temperature, moisture, humidity, and molecules in the environment that are not of interest.

25. A method comprising:
providing a sensing system comprising a first nanostructure-based sensor arranged for potential exposure to an environmental factor of interest and a second nanostructure-based sensor shielded from potential exposure to said environmental factor of interest;
exposing said sensing system to an environment; and
comparing a change in a property of the first nanostructure-based sensor with a change in a property of the second nanostructure-based sensor to determine whether the change in the property of the first nanostructure-based sensor is because of exposure to the environmental factor of interest.

26. The method of claim 25 wherein said exposing comprises:
exposing both said first and said second nanostructure-based sensors to environmental factors not of interest.

27. The method of claim 26 wherein said environmental factors not of interest include at least one of:
temperature of the environment, moisture in the environment, humidity of the environment, and gas in the environment that is not of interest.

28. The method of claim 25 further comprising:
connecting said nanostructure-based sensors to form a Wheatstone bridge, wherein the first nanostructure-based sensor is on one side of the bridge and the second nanostructure-based sensor is on an opposite side of the bridge.

29. The method of claim 28 wherein said comparing comprises:
detecting current flow across the bridge.
